(19) United States
(12) Patent Application Publication
(10) Pub. No.: US 2022/0331549 A1
(43) Pub. Date: Oct. 20, 2022

(54) BREATHING DEVICE

(71) Applicant: Nitasha Buldeo, Bedford Bedfordshire (GB)

(72) Inventor: Nitasha Buldeo, Bedford Bedfordshire (GB)

(21) Appl. No.: 17/773,013

(22) PCT Filed: Oct. 6, 2020
(86) PCT No.: PCT/GB2020/052466

§ 371 Date: Apr. 28, 2022

(30) Foreign Application Priority Data
Oct. 28, 2019 (GB) 1915620.7

(51) Int. Cl. A61M 16/20; A61M 16/06

(52) U.S. Cl. CPC A61M 16/203; A61M 16/0666; A61M 2205/3303; A61M 2016/0036

(57) ABSTRACT

A breathing device includes a first and second nasal insert for each nostril of a user. Each insert includes an opening and a valve provided at the opening for selectively restricting or allowing the passage of air through the opening. The breathing device also includes a controller adapted to control each of the valves so that the passage of air is restricted at one of the nasal inserts and allowed at the other.

The present disclosure relates to breathing devices. In particular, but not exclusively, the present disclosure relates to devices which control or assist in nasal breathing and to medical ventilators for moving, or assisting the movement of, breathable air into and out of the lungs of a patient.

### BACKGROUND

The nasal cycle is the alternating partial congestion and decongestion of the nasal cavities in humans (and other animals). At any given moment, a person will breathe through one dominant nostril; then some time later the person will switch to the other one. It is a physiological congestion of the nasal conchae due to selective activation of one half of the autonomic nervous system by the hypothalamus. Breathing through alternate nostrils showed effects on brain hemisphere symmetry on EEG topography.

It is thought that, when there is right nostril dominance, a person is generally in a more active state with an increase in heart rate, blood pressure, body temperature and with increases in cortisol, testosterone and endorphins. When there is left nostril dominance, a person is generally in a more restful state with a decrease in these factors. Alternate nostril breathing is a known yogic breath control practice. Studies have found that it can lower stress, heart rate, respiratory rate, and blood pressure. Conventionally, the practiser has to manually close and open each nostril in a consistent manner by pressing on and releasing each nostril. This can be inconvenient and distracting, particularly when the practiser is trying to meditate. Also, performing the technique properly is dependent on the skill of the practiser.

It is desirable to provide means for automatically closing and releasing the nostrils which is consistent and independent of the practiser.

It is also known to use mechanical ventilation to provide safe gas exchange, reduce the effort of breathing, improve patient-ventilator interactions, and minimize iatrogenic injury. Mechanical ventilation is indicated in individuals who are unable to sustain normal gas exchange as a result of: established or impending respiratory failure from hypoxemia, hypercapnia, or both; airway problems and to provide support to individuals undergoing general anaesthesia.

Mechanical ventilators have become more sophisticated and have expanded their application from the intensive care unit (ICU) to the respiratory medicine ward and even to patients' homes for long-term treatments. This has been the result of combining the advances in the understanding of respiratory physiology, pathophysiology and clinical management of patients together with technological progress in mechanical, electronic and biomedical engineering.

An ever-increasing number of ventilation modes and strategies are being introduced to improve outcomes, patient-ventilator interactions and patient care. A ventilator mode can be classified by specifying the control variable, breath sequence and targeting scheme. Ten maxims are commonly used to describe how to classify a given ventilation mode. The mode selected will depend on the specific needs of the individual patient.

There is now a large number of possible ventilation modes. These include conventional modes such as continuous mandatory ventilation, assist-control ventilation, intermittent mandatory ventilation, synchronised intermittent mandatory ventilation and pressure support ventilation. Alternative modes include dual control modes, such as volume-assured pressure support or pressure augmentation, volume support ventilation or pressure support ventilation, pressure-regulated volume control and auto mode ventilation. Nonconventional modes include airway pressure release ventilation, proportional assist ventilation, adaptive support ventilation, neurally adjusted ventilatory assist and high frequency ventilation including high-frequency oscillatory ventilation and high-frequency percussive ventilation.

Modern mechanical ventilators measure the ventilation variables of pressure, flow and Fraction of inspired oxygen (Fi02). The monitoring incudes measurements of peak and plateau pressures, intrinsic positive end-expiratoiy pressure and effort of breathing. These are the variables used in closed loop control of the ventilators. Heart rate variability (HRV) is variation in the time interval between heartbeats. Reduced HRV has been shown to be a predictor of mortality after myocardial infarction. HRV is commonly measured using electrocardiography. The polyvagal theory describes pathways in the autonomic nervous system that mediate HRV.

Non-invasive ventilation (NIV) is the use of breathing support administered through a face mask or nasal mask, without a need for tracheal intubation. Air, usually with added oxygen, is given through the mask under positive pressure. N1V avoids the complications of invasive ventilation such as trauma cardiac arrhythmia, hypotension, volutrauma and ventilator associated pneumonia. In conventional N1V, the same volume and frequency of air is released into both nostrils.

### SUMMARY

According to a first aspect of the present disclosure there is provided a breathing device comprising: a first and second nasal insert for each nostril of a user, each insert including an opening and a valve provided at the opening for selectively restricting or allowing the passage of air through the opening; and a controller adapted to control each of the valves so that the passage of air is restricted at one of the nasal inserts and allowed at the other.

Optionally, the controller is adapted to full close one of the valves so that the passage of air is prevented at one of the nasal inserts.

Optionally, the breathing device includes an air flow sensor provided at each of the first and second nasal inserts. Optionally, each air flow sensor is adapted to send a measurement of air flow through the respective opening to the controller.

Optionally, the controller is adapted to determine a dominant nostril associated with the greater air flow.

Optionally, the controller is adapted to restrict the passage of air at the nasal insert inserted in the dominant nostril. Optionally, the controller is adapted to periodically alternate the nasal insert in which the passage of air is restricted.

Optionally, the controller is adapted to alternate in accordance with a predetermined breathing pattern.

Optionally, the controller is a remote controller. Optionally, the controller is wirelessly connected to the first and second nasal inserts.

Optionally, the breathing device includes a first transceiver device provided at the first and second nasal inserts and a second transceiver device provided at the controller.

Optionally, the breathing device is adapted such that command signals for controlling the valves are wirelessly transmitted from the controller.

Optionally, the breathing device is adapted such that measurement signals from the air flow sensors are wirelessly transmitted to the controller.

Optionally, the controller comprises an electronic device carrying out a program. Optionally, the device comprises a smart phone, tablet, laptop or the like.

According to a second aspect of the present disclosure there is provided a ventilator apparatus comprising: a gas delivery system adapted to deliver a gas to a patient; a controller adapted to control at least one of a volume and a pressure of the delivered gas; and a sensor adapted to measure a non-ventilation variable, wherein the controller is adapted to vary the delivery of the gas based on the measured non-ventilation variable.

Optionally, the controller is adapted to vary at least one of the volume and the pressure of the delivered gas based on the measured non-ventilation variable. Alternatively, or in addition, the controller may be adapted to vary the ventilation mode based on the measured non-ventilation variable.

Optionally, the non-ventilation variable comprises an electroencephalogram (EEG) reading.

Optionally, the non-ventilation variable comprises an electrocardiogram (ECG) reading.

Optionally, the non-ventilation variable comprises a magnetoencephalogram (MEG) reading.

Optionally, the gas delivery system comprises a non-invasive ventilation (NIV) gas delivery system.

Optionally, the gas delivery system comprises a nasal insert for each nostril of the patient.

Optionally, the gas delivery system comprises a gas conduit for each nasal insert.

Optionally, the controller is adapted to individually control at least one of a volume and a pressure of the delivered gas to each nasal insert.

Optionally, the controller is adapted to vary at least one of the volume and the pressure of the delivered gas to each nasal insert based on the measured non-ventilation variable.

Optionally, the gas delivery system is adapted to deliver gas in an alternating manner to each of the nasal inserts.

Optionally, each nasal insert is expandable for maintaining the nasal insert within the nostril. Optionally, each nasal insert is inflatable.

Optionally, the sensor is provided at or near the nasal insert. Optionally, the non-ventilation variable comprises a moistness level of the or each nostril.

Optionally, the non-ventilation variable comprises an expansion level of the or each nostril.

Optionally, the non-ventilation variable comprises a pulse oximetry reading at the nostril.

Optionally, the gas delivered is air. Alternatively, the gas delivered is oxygen or oxygen enriched air.

Optionally, the apparatus is adapted to switch between a first ventilation mode and a second ventilation mode.

Optionally, the first ventilation mode comprises Assist-Control Ventilation (ACV). Optionally, the second ventilation mode comprises Synchronized Intermittent-Mandatory Ventilation (S1MV).

According to a third aspect of the present disclosure there is provided a ventilator apparatus comprising:

An N1V gas delivery system adapted to deliver a gas to a patient, the gas delivery system comprising: a nasal insert for each nostril of the patient; and a gas conduit for each nasal insert, wherein the controller is adapted to individually control at least one of a volume and a pressure of the delivered gas to each nasal insert.

Optionally, the apparatus includes a sensor adapted to measure a non-ventilation variable.

Optionally, the sensor is provided at or near the nasal insert. Optionally, the controller is adapted to vary at least one of the volume and the pressure of the delivered gas based on the measured non-ventilation variable.

Optionally, the controller is adapted to vary at least one of the volume and the pressure of the delivered gas to each nasal insert based on the measured non-ventilation variable.

Optionally, the gas delivery system is adapted to deliver gas in an alternating manner to each of the nasal inserts.

Optionally, each nasal insert is expandable for maintaining the nasal insert within the nostril. Optionally, each nasal insert is inflatable.

Optionally, the non-ventilation variable comprises a moistness level of the or each nostril.

Optionally, the non-ventilation variable comprises an expansion level of the or each nostril.

Optionally, the non-ventilation variable comprises a pulse oximetry reading at the nostril.

### BRIEF SUMMARY OF THE DRAWINGS

The present disclosure will be described below, by way of example only, with reference to the accompanying drawings, in which:

FIG. 1 is a front view of a breathing device according to a first aspect of the present disclosure;

FIG. 2 is a perspective view of a ventilator apparatus according to a second aspect of the present disclosure; and

FIG. 3 is a schematic view of a gas delivery system of the apparatus of FIG. 2. FIG. 1 shows a breathing device 60 according to a first embodiment of the present disclosure.

### DETAILED DESCRIPTION

The breathing device 60 includes a component 61 which has a first 62 and a second 64 nasal insert for insertion in each nostril of the nose 102 of a user. Each insert fits snugly in the nostril and includes an opening 66 for the passage of air during inhalation and exhalation through the nostrils by the user. A valve 68 is provided at each opening 66 for selectively restricting or allowing the passage of air through the opening 66.

The component 61 also includes an air flow sensor 70 provided at each of the first 62 and second 64 nasal inserts. Each air flow sensor 70 is adapted to measure the air flow through the respective opening 66.

The component 61 also includes a first transceiver device 80. This allows command signals for operation of each valve to be received and air flow measurements from each air flow sensor 70 to be transmitted wirelessly.

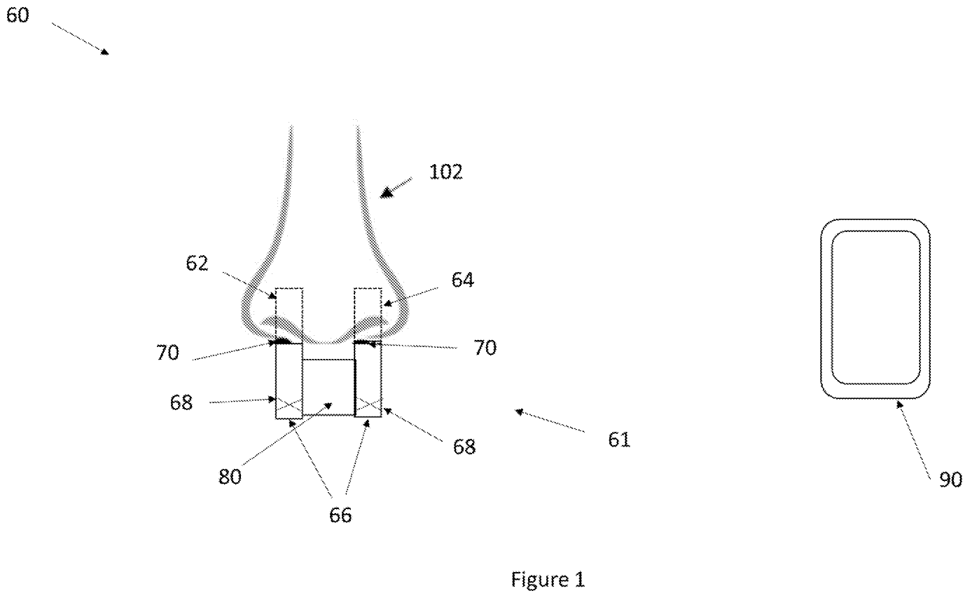

The breathing device 60 also includes a controller adapted to control each of the valves 68 so that the passage of air is restricted at one of the nasal inserts and allowed at the other. In FIG. 1, the controller 90 is provided by a smart phone 90 which is running the appropriate program or app. The smart phone 90 includes a second transceiver device for wirelessly sending command signals for operation of each valve 68 and receiving air flow measurements from each air flow sensor 70.

The app of the smart phone 90 can be adapted to perform multiple breathing patterns, each for implementing a different goal. For example, the goal may be relaxation, meditation, autonomic regulation, improved respiratory function, improved V02 max, improved sport performance or more.

The breathing pattern can be adapted to determine a dominant nostril and then take suitable action. The dominant nostril is determined using the air flow measurements from each air flow sensor 70 as the dominant nostril is associated with the greater air flow. Depending on the difference between the two air flow measurements and the desired outcome, the controller may command the valve 68 associated with the dominant nostril to partially restrict the passage of air or to fully close.

According to another breathing pattern, the controller can periodically alternate the nasal insert in which the passage of air is restricted. This can be used for alternative nostril breathing. Typically, one of the valves 68 is fully closed so that the user inhales only through one nostril. In some forms of alternative nostril breathing, the practiser should then retain the breath for a brief period. This can be assisted automatically by closing both valves 68. Then, the other valve is opened allowing exhalation through the other nostril. Then the practiser re-inhales through the other nostril, both valves 68 are then briefly closed before the initial valve is opened allowing exhalation. This whole process can be repeated a number of times for a predetermined period.

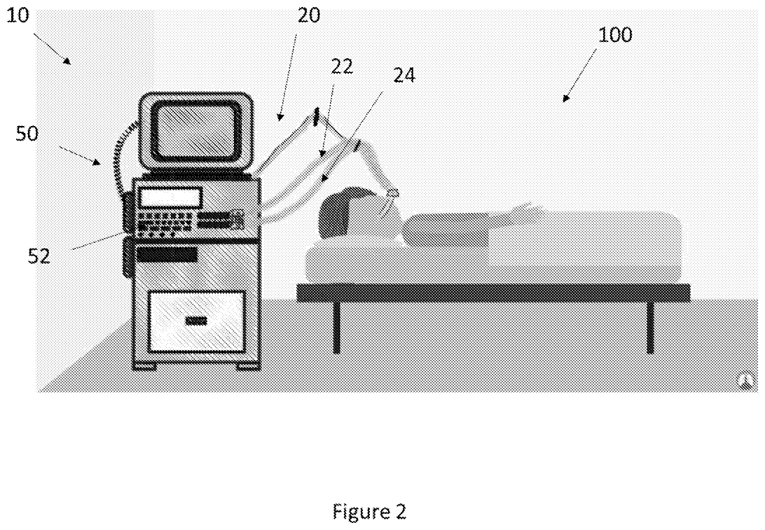

FIG. 2 shows a ventilator apparatus 10 according to a second embodiment of the present disclosure.

The apparatus 10 includes a gas delivery system 20 for delivering a gas, such as air or oxygen enriched air. This is delivered via a first gas delivery conduit 22 and a second gas delivery conduit 24 to a patient 100.

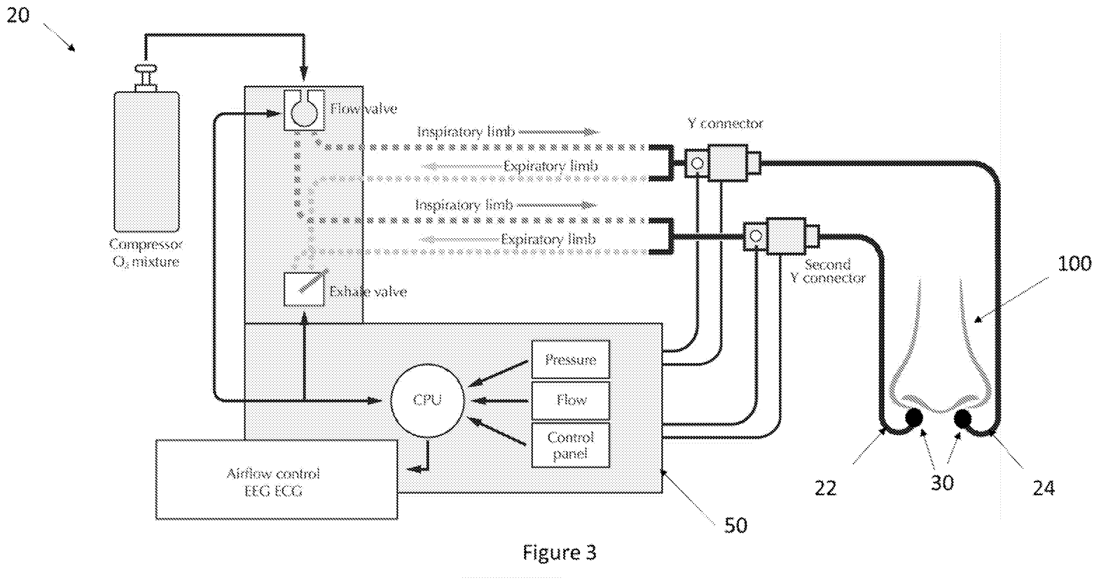

As shown in FIG. 3, each of the first gas delivery conduit 22 and the second gas delivery conduit 24 is connected to a nasal insert 30 which can be inserted into a nostril of the patient 100. Each nasal insert 30 is inflatable for forming an airtight seal within the nostril.

A sensor 40 is provided at each nasal insert 30. This is adapted to measure a non ventilation variable, in other words not a pressure or volume of the delivered gas.

The apparatus 10 also includes a controller 50 for individually controlling the volume and pressure of the gas delivered to each nasal insert 30. Input buttons 52 allow an operator to select the ventilation mode and other settings. Also, the controller 50 can vary the volume and pressure of the delivered gas based on the measured non ventilation variable. The non-ventilation variable comprises an electroencephalogram (EEG) reading. Alternative variables include an electrocardiogram (ECG) reading, a magnetoencephalogram (MEG) reading. The non-ventilation variable could also be a moistness level of each nostril, an expansion of each nostril during respiration or a pulse oximetry reading at the nostril. Each of these parameters provides an indication of the autonomic response of the patient 100. If the patient 100 is in a steady condition and breathing regularly, this represents a baseline condition for the parameter. If the patient 100 is having breathing difficulties, this parameter will vary. The controller 50 will then adjust the gas delivery settings to move the patient 100 towards the baseline condition.

For example, the apparatus 10 may start in a first ventilation mode such as ACV in which each breath is initiated by the patient but is supplemented by the apparatus 10.

If the sensor 40 measures a divergence from the baseline condition, the apparatus 10 can switch to a second ventilation mode such as S1MV in which the ventilator breaths are synchronized with patient inspiratory effort.

The apparatus 10 may also measure the gas drawn by the patient 100 via each gas delivery conduit. For example, the patient 100 may have a blockage in one nasal passage resulting in less gas being drawn via the conduit associated with this nasal passage. The apparatus 10 can be adapted to compensate for this, such as by delivering gas at a greater pressure via this conduit or by supplying a greater volume of gas via the other conduit.

The gas delivery system 20 can also be configured to deliver gas in an alternating manner to each of the nasal inserts during the inhalation phase. This simulates alternate nostril breathing and can help to lower stress, heart rate, respiratory rate, and blood pressure.

The present disclosure provides a means of controlling mechanical ventilation using the autonomic response of the patient 100. This can be used to respond to irregularities which are not easily detected by measuring ventilation variables. Ventilation can be optimised through individual control of gas delivery.

The present disclosure has particularly beneficial applications for cardiac rehabilitation, post cardiac surgery recovery, and autonomic dysfunction.

Various modifications and improvements can be made to the above without departing from the scope of the present disclosure.

What is claimed is:

1-38. (canceled)

39. A breathing device comprising:
a first nasal insert and a second nasal insert for each respective nostril of a user, each of the first nasal insert and the second nasal including an opening and a valve provided at the opening for selectively restricting or allowing a passage of air through the opening;
a controller configured to control each of the valves so that the passage of air is restricted at one of the first and second nasal inserts and allowed at the other; and
an air flow sensor provided at each of the first and second nasal inserts.

40. The breathing device as claimed in claim 39, wherein the controller is configured to fully close one of the valves so that the passage of air is prevented at one of the first and second nasal inserts.

41. The breathing device as claimed in claim 40, wherein the controller is configured to determine a dominant nostril associated with the greater air flow.

42. The breathing device as claimed in claim 41, wherein the controller is configured to restrict the passage of air at the first or second nasal insert inserted in a dominant nostril.

43. The breathing device as claimed in claim 39, wherein the controller is configured to periodically alternate the frist or second nasal insert in which the passage of air is restricted.

44. The breathing device as claimed in claim 43, wherein the controller is configured to alternate in accordance with a predetermined breathing pattern.

45. The breathing device as claimed in claim 39, wherein the controller is wirelessly connected to the first and second nasal inserts.

46. The breathing device as claimed in claim 45, wherein the breathing device includes a first transceiver device provided at the first and second nasal inserts and a second transceiver device provided at the controller.

47. The breathing device as claimed in claim 45, wherein the breathing device is configured such that command signals for controlling the valves are wirelessly transmitted from the controller.

48. The breathing device as claimed in claim 45, wherein the breathing device is configured such that measurement signals from the air flow sensors are wirelessly transmitted to the controller.

49. The breathing device as claimed in claim 39, wherein the controller comprises an electronic device programmed for carrying out a program.

50. The breathing device as claimed in claim 49, wherein the electronic device comprises a smart phone, tablet or laptop.

51. A ventilator apparatus comprising:
a gas delivery system configured to deliver a gas to a patient;
a controller configured to control at least one of a volume and a pressure of the delivered gas; and
a sensor configured to measure a non-ventilation variable, the controller being configured to vary the delivery of the gas based on the measured non-ventilation variable.

52. The apparatus as claimed in claim 51, wherein the controller is configured to vary at least one of the volume and the pressure of the delivered gas based on the measured non-ventilation variable.

53. The apparatus as claimed in claim 51, wherein the controller is configured to vary a ventilation mode based on the measured non-ventilation variable.

54. The apparatus as claimed in claim 51, wherein the non-ventilation variable comprises at least one of an electroencephalogram (EEG) reading, an electrocardiogram (ECG) reading, and a magnetoencephalogram (MEG) reading.

55. The apparatus as claimed in claim 51, wherein the gas delivery system comprises a non-invasive ventilation (NIV) gas delivery system.

56. A ventilator apparatus comprising:
a non-invasive ventilation (NIV) gas delivery system configured to deliver a gas to a patient, the gas delivery system comprising:
a nasal insert for each nostril of the patient; and
a gas conduit for each nasal insert,
the controller being configured to individually control at least one of a volume and a pressure of the delivered gas to each nasal insert.

57. The apparatus as claimed in claim 56, further comprising a sensor for each nasal insert configured to measure a non-ventilation variable, and wherein the sensor is provided at or near the respective nasal insert.

58. The apparatus as claimed in claim 57, wherein the controller is configured to vary at least one of the volume and the pressure of the delivered gas based on the measured non-ventilation variable.
